(19) United States
(12) Patent Application Publication
(10) Pub. No.: US 2010/0125335 A1
(43) Pub. Date: May 20, 2010

(54) METHODS AND APPARATUS FOR REPLACING BIOLOGICAL JOINTS USING BONE CEMENT IN A SUSPENDED STATE

(76) Inventor: Robert J. Daley, Hinsdale, IL (US)

Correspondence Address: K&L Gates LLP, P.O. Box 1135, CHICAGO, IL, 60690, US

(21) Appl. No.: 12/544,697

(22) Filed: Aug. 20, 2009

Related U.S. Application Data
(60) Provisional application No. 61/116,536, filed on Nov. 20, 2008.

(51) Int. Cl. A61F 2/30; A61L 27/14

(52) U.S. Cl. 623/18.11; 523/116

(57) ABSTRACT

The present disclosure provides methods and apparatus for replacing biological joints, but applies also to the fixation of any solid implant for use in dental or orthopaedic applications. In general, an ideal amount of bone cement is applied to the implant prior to going in to the operating room. Next, the polymerization (e.g., drying) process is suspended with a coating and/or a chemical. Once the implant(s) are needed in the operating room, the polymerization process is resumed. In this manner, the bone cement does not need to be mixed in the operating room, the surgeon does not need to “race the clock,” each implant is placed with the ideal amount of cement in the ideal consistency, there are no powder clumps, and there is little to no excess cement to remove.

### PRIORITY CLAIM

This application claims the benefit of U.S. Provisional Patent Application No. 61/116,536, filed Nov. 20, 2008, entitled “Methods and Apparatus for Replacing Biological Joints,” the entire contents of which are hereby incorporated by reference.

### TECHNICAL FIELD

The present application relates in general to joint replacement surgery and more specifically to methods and apparatus for replacing biological joints, but applies also to the fixation of any solid implant for use in dental or orthopaedic applications.

### BACKGROUND

When a surgeon performs a joint replacement (e.g., a hip replacement), he must attach one or more implants to one or more bones. For example, in a total knee replacement, the surgeon typically attaches two or three different implants to two or three different bones. These implants are typically made of metal, plastic, or ceramic in any combination and are attached in one of two ways.

Using one attachment method, each implant is attached to the bone using bone cement. Bone cement is typically an acrylic material dispensed as a powder and a liquid, that is mixed as is any other cement. The powder contains polymethyl methacrylate, (or similar type material) a filler, plasticizer, and polymerization initiator. The liquid monomer may be methyl methacrylate with an inhibitor and an activator.

This method is problematic because the bone cement must be mixed and applied in the operating room. This requires know-how and skill that is not always present in the assistants during the operation. The mixing in the operating room also lengthens the time for the operation as the mixing process takes time to set up and perform. In addition, the time needed for application of the cement and cleaning the excess cement lengths the operation time. After the bone cement is mixed, the surgeon must “race the clock” as the cement starts to dry. For example, bone cement may be applied to one implant, which is then placed in the patient and excess cement is cleaned off. Next, bone cement may be applied to another implant, which is then placed in the patient and excess cement is cleaned off. In some cases, additional round(s) of applying bone cement, placing implant(s) and cleaning of the excess cement are required. If more than one implant is to be placed in the patient, the polymerization phase of the cement (e.g., the hardness of the cement from liquid to solid) is different for each implant. In other words the bone cement is more polymerized and firmer for later placed implants. As a result, each implant is not placed with the bone cement at the ideal consistency. In addition, if there are any powder clumps, in the cement from improper mixing by the technician, complications can occur (e.g. crack propagation from the voids in the cement mantle). Finally, after the bone cement is injected, the excess must be cleaned out. If any cement is missed, that excess cement may cause wearing complications for the artificial joint and the patient by interposing third body wear and causing abrasive wear to the implant.

Using another attachment method, each implant is manufactured with a rough contact surface such as a porous surface. This roughened surface may also be coated with a very thin bone mineral substance layer, e.g. hydroxyapatite or other calcium mineral substances, that is applied and is hardened into a solid layer on the implant substrate. The bone grows into this layer, and no bone cement is needed.

However, this “cementless” type of operation may not be successful if micromotion during healing interferes with this bone ingrowth into the roughened layer of the prosthesis. This lack of ingrowth could lead to a painful joint because of the lack of bony fixation. One potential solution is to apply a layer of bone mineral substance hardened to the prosthesis to increase the healing speed and consistency of bony ingrowth. However this does not address the initial fixation problem, leaving this still susceptible to micromotion and with that a lack of bony ingrowth and with it pain. Another alternative is to apply a paste of bone mineral substance on at the time of implantation. However, this has some of the same problems as applying bone cement. Namely, the surgeon must “race the clock” as the calcium layer hardens.

### SUMMARY

The present disclosure provides methods and apparatus for replacing biological joints that solve all of these problems. As described in more detail below, an ideal amount of factory mixed bone cement is applied to the implant prior to going in to the operating room. Next, the polymerization (e.g., hardening) process is suspended with the cement in a semi solid-liquid stage, e.g. doughy state, by a manufacturing process such as a coating and/or a chemical. For example, the polymerization process may be suspended and then resumed using by freezing and then thawing the bone cement. Once the implant(s) are needed in the operating room, the polymerization process is resumed. In this manner, the bone cement does not need to be mixed in the operating room, and the surgeon does not need to “race the clock.” Each implant is placed with the ideal amount of cement in the ideal consistency, in the ideal distribution on the implant, there are no powder clumps, and there is little to no excess cement to remove. The surgeon is able to open the implant package and implant the prosthesis with confidence that fixation is achieved without the inherent problems of cement mixing.

### BRIEF DESCRIPTION OF THE FIGURES

FIG. 1 is a flowchart of an example method of replacing a biological joint.

FIG. 2 is diagram illustrating a portion of an implant including bone cement in a pre-suspended polymerization state.

FIG. 3 is diagram illustrating a portion of an implant including bone cement in a suspended polymerization state.

FIG. 4 is diagram illustrating a portion of an implant including bone cement in a resumed polymerization state.

### DETAILED DESCRIPTION

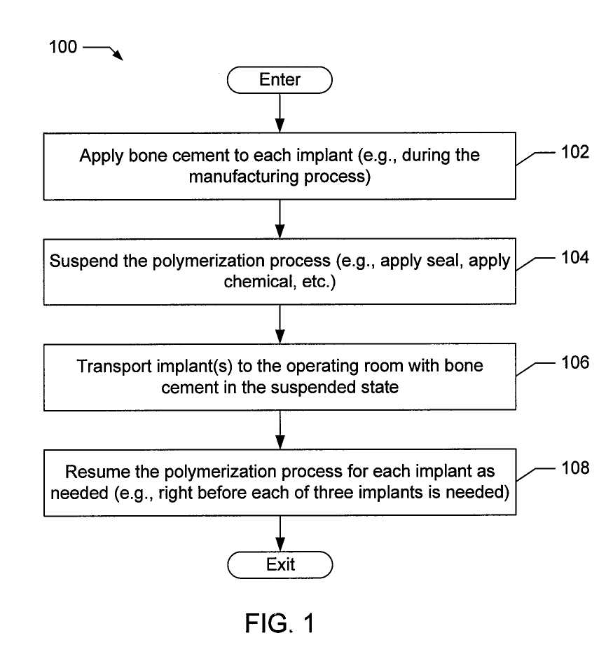

Turning to the figures, a flowchart of an example process 100 for replacing biological joints is presented in FIG. 1. Although the process 100 is described with reference to the flowchart illustrated in FIG. 1, it will be appreciated that many other methods of performing the acts associated with process 100 may be used. For example, the order of many of the steps may be changed, some of the steps described may be optional, and additional steps may be included. In addition, it will be appreciated that the methods disclosed herein also apply to the fixation of any solid implant for use in dental or orthopaedic applications.

In general, during the process 100, an ideal amount of bone cement is applied in the desired location on the implant prior to going in to the operating room. Next, the polymerization (e.g., drying) process is suspended with a manufacturing process, coating and/or a chemical. Once the implant(s) are needed in the operating room, the polymerization process is resumed.

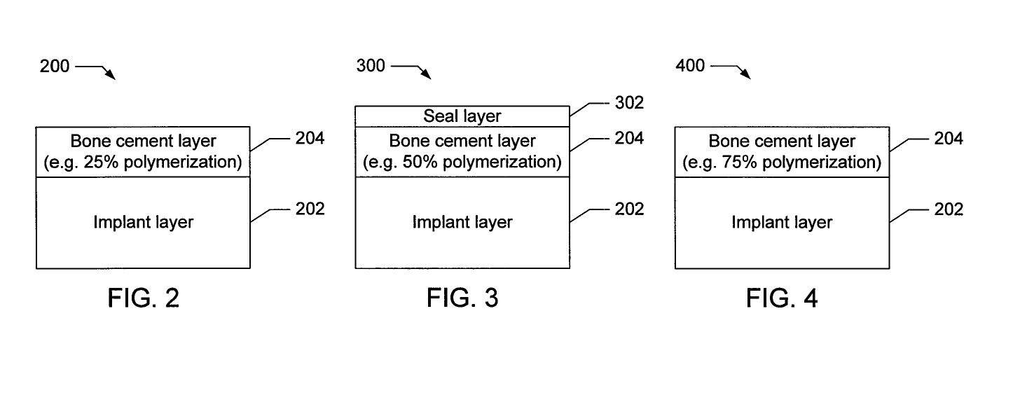

The process 100 begins when an ideal amount of bone cement is applied to the implant prior to going in to the operating room (block 102). An example of a portion 200 of an implant 202 including bone cement 204 in such a pre-suspended polymerization state (e.g., 25% polymerization) is illustrated in FIG. 2. For example, the bone cement may be applied in the factory.

Next, the polymerization process is suspended with a manufacturing process, coating and/or a chemical (block 104). An example of a portion 300 of an implant including bone cement in such a suspended polymerization state (e.g., 50% polymerization) is illustrated in FIG. 3. For example, a plastic seal similar to plastic food wrap, such as polyvinylidene chloride or low density polyethylene, may be used to prevent air from reaching the bone cement. Alternatively, the entire end of the implant may be submerged in a chemical to suspend the polymerization process.

Once the implant(s) are transported to the operating room (block 106), the polymerization process is resumed (block 108). An example of a portion 400 of an implant including bone cement in such a resumed polymerization state (e.g., 75% polymerization) is illustrated in FIG. 4. For example, the surgeon or an assistant may peel off a plastic coating in the operating room right before each implant is attached to the patient. Alternatively, the entire end of the implant may be submerged in a chemical to resume the polymerization process.

In this manner, the bone cement does not need to be mixed in the operating room, the surgeon does not need to “race the clock,” each implant is placed with the cement in the ideal consistency, there are no powder clumps, and there is little to no excess cement to remove.

In summary, persons of ordinary skill in the art will readily appreciate that methods and apparatus for replacing a biological joint have been provided. The foregoing description has been presented for the purposes of illustration and description. It is not intended to be exhaustive or to limit the invention to the exemplary embodiments disclosed. Many modifications and variations are possible in light of the above teachings. It is intended that the scope of the invention be limited not by this detailed description of examples, but rather by the claims appended hereto.

What is claimed is:

1. A method of replacing a biological joint, the method comprising:
applying bone cement to an implant, the bone cement being associated with an active polymerization process;
placing the polymerization process into a suspended state;
transporting the implant to an operating room with the bone cement in the suspended state; and
resuming the polymerization process in the operating room.

2. The method of claim 1, wherein the bone cement includes an acrylic material that is produced by mixing a powder and a liquid.

3. The method of claim 2, wherein the powder includes a polymethyl methacrylate, a filler, a plasticizer, and a polymerization initiator.

4. The method of claim 2, wherein the liquid includes methyl methacrylate with an inhibitor and an activator.

5. The method of claim 1, wherein the implant is made of at least one of metal, plastic, and ceramic.

6. The method of claim 1, wherein placing the polymerization process into a suspended state includes covering the bone cement with a coating.

7. The method of claim 6, wherein the coating includes a plastic seal.

8. The method of claim 6, wherein the coating includes polyvinylidene chloride.

9. The method of claim 6, wherein the coating includes polyethylene.

10. The method of claim 1, wherein placing the polymerization process into a suspended state includes adding a chemical to the bone cement.

11. The method of claim 1, wherein placing the polymerization process into a suspended state includes freezing the bone cement.

12. The method of claim 1, wherein resuming the polymerization process includes removing a coating from the bone cement.

13. The method of claim 1, wherein resuming the polymerization process includes adding a chemical to the bone cement.

14. The method of claim 1, wherein resuming the polymerization process includes thawing the bone cement.

15. The method of claim 1, wherein the implant includes a hip implant.

16. The method of claim 1, wherein the implant includes a knee implant.

17. The method of claim 1, wherein the implant includes a dental implant.

18. The method of claim 1, wherein the implant includes an orthopaedic implant.

19. A joint replacement apparatus comprising:
an implant; and
bone cement attached to the implant, the bone cement being associated with an active polymerization process that is in a suspended state.

20. The apparatus of claim 19, wherein the bone cement includes an acrylic material that is produced by mixing a powder and a liquid.

21. The apparatus of claim 20, wherein the powder includes a polymethyl methacrylate, a filler, a plasticizer, and a polymerization initiator.

22. The apparatus of claim 20, wherein the liquid includes methyl methacrylate with an inhibitor and an activator.

23. The apparatus of claim 19, wherein the implant is made of at least one of metal, plastic, and ceramic.

24. The apparatus of claim 19, including a coating over the bone cement to place the bone cement in the suspended state.

25. The apparatus of claim 24, wherein the coating includes a plastic seal.

26. The apparatus of claim 24, wherein the coating includes polyvinylidene chloride.

27. The apparatus of claim 24, wherein the coating includes polyethylene.

28. The apparatus of claim 19, including a chemical over the bone cement to place the bone cement in the suspended state.
